(19) United States
(12) Patent Application Publication
(10) Pub. No.: US 2009/0253134 A1
(43) Pub. Date: Oct. 8, 2009

(54) Cellular Pyrogen Test

(75) Inventors: Herwig Brunner, Stuttgart (DE); Doris Finkelmeier, Stuttgart (DE); Georg Geiger, Böblingen (DE); Anke Burger-Kentischer, Stuttgart (DE)

Correspondence Address: BATEMAN IP LAW GROUP, P.O. BOX 1319, SALT LAKE CITY, UT, 84110, US

(73) Assignee: Fraunhofer-Gesellschaft zur Forderung der angewandten Forschung e.V., Munchen (DE)

(21) Appl. No.: 12/307,711

(22) PCT Filed: Jul. 5, 2007
(86) PCT No.: PCT/EP07/05946

§ 371 Date: Jan. 6, 2009

(30) Foreign Application Priority Data
Jul. 7, 2006 (DE) 10 2006 031 483.2

(51) Int. Cl. C12Q 1/68; C12N 5/10; C12Q 1/02; C12Q 1/42; C12Q 1/66

(52) U.S. Cl. 435/6; 435/352; 435/29; 435/404; 435/21; 435/18; 435/8

(57) ABSTRACT

The invention concerns methods, agents and kits for qualitative and quantitative detection and identification of pathogens and pathogen spectra based on endotoxins and other pyrogens.

### RELATED APPLICATIONS

The present application is the U.S. National Phase of PCT Application PCT/EP2007/005946, filed Jul. 5, 2007, claiming priority to German Patent Application No. 10 2006 031 483.2, filed Jul. 7, 2006.

### BRIEF SUMMARY OF THE INVENTION

The invention concerns methods, agents and kits for qualitative and quantitative detection and identification of pathogens and pathogen spectra based on endotoxins and other pyrogens.

### BACKGROUND OF THE INVENTION

Rapid and reliable identification of pathogen spectra is of great significance in clinical diagnosis in hospitals for initiation of targeted infection therapy, for example, in sepsis patients.

Sepsis and multiorgan failure associated with sepsis are the most important mortality factors worldwide and are among the unsolved problems of medicine. According to conservative estimates about 500,000 patients still die annually worldwide as a result of “blood poisoning,” which amounts to 1400 per day. The annual burden on the health budget by treatment of patients with serious sepsis was estimated at 17 million dollars in the US. The sum of life-threatening disease symptoms and pathophysiological changes is referred to as sepsis (septicemia, blood poisoning). It is caused by pathogenic germs and their products which penetrate into the blood stream from a focus of infection. The immune reaction that sets in as a result leads to the formation of endogenous mediators (cytokines). This activates the inflammation cascade and a systemic inflammatory reaction that can no longer be controlled is the result. Despite intensive care measures, the prognosis is serious and the mortality is about 50%. The prognosis is particularly unfavorable with late onset of therapy, an unlocalizable focus of infection or an unidentifiable pathogen.

Pathogens that trigger sepsis are generally bacteria, mostly Gram-negative bacteria, like E. coli, other enterobacteria, Klebsiella, Proteus, Enterobacter species, Pseudomonas aeruginosa, Neisseria meningitidis and Bacteroides, but also common Gram-positive bacteria like Staphylococcus aureus, Streptococcus pneumoniae and other streptococci; rare fungi, viruses or parasites (bacteremia, fungemia, viremia, parasitemia). Excretion of endogenous mediators, like interleukins, tumor necrosis factors, histamine, serotonin, oxygen radicals and proteases is stimulated by release of so-called microbial structures (for example, endotoxins, exotoxins, superantigens). By activation of leukocytes and humoral defense systems they lead to the changes typical of septic shock. Systemic inflammation as a reaction to circulating microbial antigens is an important characteristic of the pathophysiology of sepsis and septic shock. On contact of cells of nonspecific immune defense with lipopolysaccharide (LPS), components of the bacterial cell wall, peptidoglycans or lipoteichonic acid (LTA), natural immunity is activated and in the early phase of infection cytokines are secreted by different immune cells. Although these cytokines play an important role in the defense reaction, activated neutrophils, for example, are lured to the location of inflammation, the entry of the cytokines and bacterial substances into the blood stream leads to a chain of unfavorable pathophysiological events. The clinical symptom complex accompanying this inflammation reaction is referred to as systemic inflammatory response system (SIRS).

In the presence of a septic disease or even on suspicion of such a disease, therapy must occur in timely fashion. Thus far there has been no opportunity to quickly and reliably identify the pathogen spectrum of a sepsis patient. The usual diagnosis of sepsis consists generally of repeated taking of a clinical sample: blood and urine culture, sputum, stool, wound secretions for pathogen identification with resistance determination before the beginning of antibiotic therapy. The probability of pathogen detection with previous methods in septicemia is 30 to 50%. This can only be achieved by setting up several cultures for microbiological investigation, germ culturing experiments from a venous blood sample or from urine. The sample is inoculated and incubated in a liquid nutrient medium. This method costs valuable time, often does not lead to identification of the pathogen, since it is only possible in vital pathogens. If the sample was taken during antibiotic therapy, the culturing experiments are generally unsuccessful. Consequently, broadly conceived antibiotic, antimycotic, antiviral and/or antiparasite therapy is still used. Pyrogenic substances of the pathogens, like cell wall components, cannot be detected with this method.

Consequently there is a demand for a rapid and simple test system that permits detection and differentiation of the sepsis pathogen or pathogen spectrum.

Pyrogens are fever-producing substances, so-called pyrogenic substances that induce endogenous cells capable of phagocytosis (immune cells) to synthesize proinflammatory interleukins (mostly IL-1 and IL-6) and tumor necrosis factor α (TNF-α) which then influence the temperature center of the body as “intrinsic pyrogens” so that increased heat production and reduced heat release occur. The most strongly active pyrogens originate from Gram-negative bacteria. The pyrogens are not a uniform substance group. They include cell wall components and metabolic products of microorganisms (apathogenic and pathogenic bacteria, fungi and viruses) as well as parasites, for example, endotoxins, exotoxins or superantigens.

Pyrogens are mostly of clinical significance during injection or infusion of pyrogen-containing liquids, like stabilizer solutions, during use of bacterially contaminated banked blood, nonpyrogen-free injection syringes, infusion equipment, etc. The lack of apyrogenicity is the main cause for so-called “transfusion incidents,” which are accompanied by high fever, shock, consumption coagulopathy and acute kidney failure. Especially today, additional risk factors via which pyrogens can reach the body include central venous catheters, long-term tube feeding and long-term ventilation.

Pyrogens are generally heat-resistant and dialyzable substances, for example, lipopolysaccharide-protein-lipid complexes, LPS. Ordinary methods for sterilization of infusion solutions, instruments and equipment intended for use on the human or animal body are therefore not sufficient to eliminate these pyrogenic substances. Additional cleaning steps are essential. Apyrogenicity is an essential condition for use of such products in the body. All products that come into intense contact with the human or animal body, either because they are administered into the blood stream or because they spend a long time in the body, should also be sufficiently apyrogenic.

The spectrum of occurring pyrogenic substances depends on the pathogen or pathogen spectrum. Pathogens form pathogen-typical or pathogen-specific pyrogen patterns, so-called pathogen-associated microbial patterns or PAMPs. A classification of a certain pathogen or pathogen spectrum could occur by identification and differentiation of PAMPs.

In order to detect pyrogens or PAMPs in a sample (pyrogen test) mostly three commercially employed detection methods or tests are now available. One known test is the rabbit pyrogen test. It is based on the “fever reaction” of animals to pyrogens. This is an animal experiment in which the rabbit is administered the test substance in the ear vein. To detect a defense reaction of the animal body to the substance, rectal fever is measured after several hours. This test is time-consuming and cost-intensive and connected with calculated suffering of animals. Endotoxin and non-endotoxin pyrogens can be detected with it but not identified. A test for viruses is not possible. Specification of the PAMPs is not possible. Its transferability to humans is also disputed.

Another known test is the Limulus amebocyte lysate test (LAL) (for example, Cambrex Bioscience or Charles River Co.). It is based on the defense reaction of arthropods to certain substances known as pyrogens. In the LAL a proenzyme is recovered from the blood cells of Limulus polyphemus, which is converted to an active enzyme via a Gram-negative bacterial endotoxin. The amount of endotoxin can be determined quantitatively, for example, by means of a photometer by an enzyme substrate conversion. This method is more sensitive and better standardizable than the known rabbit test but records only endotoxins of Gram-negative organisms (for example, lipopolysaccharide LPS; detection limit: 3 pg/mL). Such endotoxins represent only a small fraction of known pyrogenic substances. Other pyrogens remain unrecognized. In recent years, however, Gram-positive pathogens have gained increasing significance relative to the Gram-negative bacteria.

Another known test is finally the immune pyrogen test, for example Endosafe IPT (Charles River Co.). It is based on the fever reaction of human cells to pyrogens that are present. This is a human whole blood test in which the cytokine IL-1 is excreted as a response to a pyrogenic substance from vital blood cells, which can be determined quantitatively by means of ELISA (detection limit: 20-50 pg/mL). This system also records pyrogens of Gram-positive pathogens. The test, however, is still connected with greater time and work demands. Human whole blood must be prepared, which is potentially pathogenic. A specification of PAMPs is not possible.

The known tests are time-consuming and require a well-equipped laboratory (ELISA test, human blood processing, animal experiment). There is consequently a demand for a rapid test system that can be conducted simply for detection of pyrogens. There is also a demand for a test system for specification of the pyrogen pattern PAMP in order to be able to draw conclusions concerning the pathogen or pathogen spectrum. This is advantageous for diagnosis and treatment of infectious diseases in which the occurrence of pyrogens plays a role, especially sepsis.

There is a demand for improved tests on pyrogen residues on medical equipment, donor tissue, injectable drugs and medical products, like implants or instruments (catheters, etc.). There is also a demand in the food industry and pharmaceutical industry for improved detection of pyrogenic substances and germs and their identification in foods, food ingredients, raw materials and starting materials for foods or drugs.

It is known that so-called toll-like receptors (TLR, TLRs) are connected with pathophysiological processes in sepsis and similar infectious diseases accompanied by the occurrence of pyrogens in the body. TLRs mediate the endogenous reactions to pyrogens. In microbially triggered sepsis bacterial components stimulate the immune cells of the host via the TLRs.

TLRs are highly preserved transmembrane proteins with leucine-rich extracellular domains and a cytoplasmic domain of about 200 amino acids. Because of their homology in the cytoplasm domain they belong to the interleukin-1 receptor/toll-like receptor superfamily. The characteristic cytoplasmic TIR domain is essential for signal transmission. The extracellular domain directly participates in recognition of the different pathogenic molecular structures and differs sharply from that of the IL-1 receptor. Whereas the extracellular part of the IL-1 receptor consists of three immunoglobulin domains, TLRs possess 18 to 26 LRR each 24 to 29 amino acids long. In contrast to the protein “toll” known from Drosophila, TLRs are directly activated by foreign structures. Thus far 10 different human TLRs and 13 TLRs of the mouse have been identified. They are expressed on different cell types of the immune system, mostly monocytes, macrophages, dendritic cells, as well as B and T cells. TLRs are located on the plasma membrane; TLR-3, TLR-7 and TLR-9 are activated by nucleic acid motifs and can be found in intracellular compartments.

TLR-2 is essential for recognition of a number of PAMPs from Gram-positive bacteria, including bacterial lipoproteins and lipoteichonic acids. TLR-3 is involved in the recognition of double-stranded viral RNA. TLR-4 is mostly activated by LPS. TLR-5 detects bacterial flagellin. TLR-7 and TLR-8 recognize synthetic small antiviral molecules and single-stranded RNA. TLR-9 was detected in endoplasmic reticulum (ER) and after stimulation with DNA containing CpG motifs, for example, CpG oligodeoxynucleotides, is recruited into the endosomal/lysosomal compartments. CpG motifs are areas with a nucleic acid strand in which the components cytosine (C) and guanine (G) occur with unexpected frequency (“p” stands for a phosphate group that joins both components “C” and “G”); such CpG motifs are found particularly often in the genome of bacteria and viruses, but not vertebrates.

Antagonists of the toll-like receptors are being increasingly used in dermatology, for example, to treat virus-induced papillomas. There is a demand for a test system for screening of new TLR antagonists.

The concept of activation of the human immune system is of interest in cancer therapy. Substances, like CPG 7909 (Coley Pharmaceutical Group), cause immune-modulatory effects in this way and can therefore improve the efficacy of chemotherapies. There is a demand for a test system for screening of new CpG motifs (oligodeoxynucleotides).

### DETAILED DESCRIPTION

The present invention is based mostly on the technical problem of providing methods and agents for specific detection of pyrogens (specific pyrogen test). Another technical problem is connected with it in the preparation of methods and agents for a specific detection of pathogens or pathogen spectra in infections of the human or animal body. Another technical problem is connected with it in the preparation of methods and agents for screening of new TLR antagonists and/or new CpG motifs.

The technical problem is essentially solved by the preparation of a transgenic cell or cell line for specific detection of a pyrogen in a sample with the characterizing features according to claim 1. The cell is preferably adherent. In one variant the cell is preferably in a suspension.

According to the invention the transgenic cell or cell line in the genome has (a) at least one gene or genes that code for at least one toll-like receptor (TLR), and (b) at least one reporter gene, which is under the expression control of a promoter inducible by NF-κB. The cell or cell line in the genome preferably also has a gene that codes for the CD14 receptor. The cell or cell line according to the invention is preferably based on a fibroblast cell, preferably mammal fibroblast cells, especially the murine fibroblast cells of the type NIH-3T3. The TLR is preferably transfected together with the coreceptor CD14 (MD2) via plasmids.

The invention therefore proposes to furnish a transgenic cell line, preferably based on the fibroblast cells NIH-3T3, which expresses at least one TLR and preferably co-expresses the CD14 coreceptor. Co-expression of TLR-4 and CD14 is particularly preferred. The inventors surprisingly found that this transgenic cell, in contact with pyrogens that specifically activate the expressed TLR, expresses enzyme activity coded by the receptor gene, which can be detected, for example, by color reaction and quantified under certain conditions. A cellular test system is therefore provided for detection of pyrogens, PAMPs and other TLR-activating substances.

The selectivity and sensitivity of this test system is high. The sensitivity is about 1 to 10 pg/mL LPS. In comparison with this the sensitivity of ordinary pyrogen test systems is about 3 to 10 pg/mL (LAL) or 20-50 pg/mL (IPT).

The test system according to the invention can get by without the equipment of a cell culture laboratory, like CO₂ gassing, etc. and can therefore be used simply for any user even without special laboratory equipment.

The principal test methods characterized by the teachings according to the invention can be expanded to cell lines for all TLRs (for example, human TLRs 1-10) so that all PAMPs can be selectively recognized and identified with it. A simple and rapid cellular test system can thus be advantageously furnished, which permits specific detection of one or more pyrogens or (pathogen-associated microbial patterns) PAMPs as well as their quantification. Without being bound to a theory, activation of TLRs induces signal transduction pathways that lead to production of different cytokines by means of the transcription factor NF-κB. A number of proteins are involved in the signal cascade, like MyD88 and IRAK1. This signal cascade leads to induction and production of pro-inflammatory cytokines via activation of the transcription factor NF-κB. The cytokines tumor necrosis factor (TNF), interleukin-1 (IL-1) and interleukin-6 (IL-6) are considered the most important centrally and initially involved mediators in this process. After activation of TLRs, recruiting of adapter molecules and production of pro-inflammatory cytokines occur. Secretion of these cytokines leads to stimulation of the immune system and defends against the penetrating microorganism. However, sharply overshooting production can lead to sepsis or septic shock. In conjunction with diagnosis of sepsis mostly TLR-2 and TLR-4 are preferred. Whereas TLR-2 recognizes components of Gram-positive bacteria, like peptidoglycans, lipopeptides and LTA, TLR-4 is the receptor for LPS, the main ingredient of the cell wall of Gram-negative bacteria. TLR-2 and TLR-4 are therefore prominent in Gram-positive and Gram-negative sepsis as signal-transmitting receptors and should therefore be preferably used for differentiation of the pathogen spectrum. Table 1 shows the specificities of the individual human TLRs.

TABLE 1

| Ligand/Pyrogen | Origin | TLR type |
|---|---|---|
| Cell wall components | Bacteria | TLR 1/TLR 2 |
| (peptidoglycan, lipopeptide, | | (heterodimer) |
| lipoteichonic acid | | |
| Lipoproteins | Bacteria | TLR 2 |
| Lipopeptide | Mycoplasmas | TLR 2/TLR 6 |
| | | (heterodimer) |
| Zymosan | Yeasts, fungi | TLR 2/TLR 6 |
| | | (heterodimer) |
| Double-stranded RNA | Viruses | TLR 3 |
| Lipopolysaccharides (LPS) | Gram-negative | TLR 4, CD 14 |
| | bacteria | |
| Heat-shock protein 60 (Hsp 60) | Human/fungi | TLR 4 |
| Flagellin | Bacteria | TLR 5 |
| Single-stranded RNA | Viruses | TLR 7/TLR 8 |
| | | (heterodimer) |
| Unmethylated CpG motifs | Bacteria, viruses | TLR 9 |

By identification of the pathogen-specific PAMPs the possibility of rapid identification of sepsis-triggering germs is obtained. Sepsis patients can be treated in timely and targeted fashion with the cellular test system according to the invention.

In a preferred variant it is proposed that the transgenic cell co-express at least two different TLRs so that formation of TLR heterodimers occurs, which have their own specificity (see Table 1). The cell therefore preferably has a gene or genes that code for a first toll-like receptor type (TLR type) and additionally a gene or genes that code for a second toll-like receptor type (TLR type).

A “reporter gene” is understood to mean one or more genes or gene constructs that code for enzyme activity under the control of an inducible promoter, which is not constitutively expressed or only insignificantly so in the host organism. The occurrence of coded enzyme activity indicates induction of the reporter gene promoter. The reporter gene and inducible promoter preferably lie on a reporter gene plasmid. It is proposed to induce the reporter gene promoter by a transcription factor, which, without being bound to the theory, is a component of the TLR-induced intracellular signal cascade. The proposed at least one reporter gene according to the invention is preferably under the control of the transcription factor “nuclear factor kappa-B” (NF-κB). On activation of TLR, bonded NF-κB localized in the cytoplasm is released and translocated into the cell nucleus. A preferred NF-κB inducible promoter is selectin or ELAM-1 (endothelial cell leukocyte adhesion molecule-1) promoter.

A preferred reporter gene is the SEAP (secreted alkaline phosphatase), preferably under control of the ELAM-1 promoter, preferably in the form of a reporter gene plasmid. Another preferred reporter gene is the P-galactosidase gene lacZ, preferably under control of the ELAM-1 promoter, preferably in the form of a reporter gene plasmid. Another preferred reporter gene is the luciferase gene, preferably under control of the ELAM-1 promoter, preferably in the form of a reporter gene plasmid. Another preferred reporter gene is GFP (green fluorescent protein), preferably under control of the ELAM-1 promoter, preferably in the form of a reporter gene plasmid. Any other promoter suitable for the corresponding application can naturally be used, which has the property of being modulated by a signal cascade triggered by activation or bonding of TLR.

The TLR is preferably chosen from the ten now known human TLRs. It is understood that the invention is not restricted to the known human TLRs. Additional TLRs still to be designated are included in the present invention. Thus, another object according to the invention is a transgenic cell or cell line that has at least one gene of a still not further designated TLR variant and expresses this TLR variant.

In a preferred variant of the invention the cell or cell line expresses at least the human TLR type 1 (TLR-1). In another preferred variant the cell or cell line expresses at least the human TLR type 2 (TLR-2). In another preferred variant the cell or cell line expresses at least the human TLR type 3 (TLR-3). In another preferred variant the cell or cell line expresses at least the human TLR type 4 (TLR-4). In another preferred variant the cell or cell line expresses at least the human TLR type 5 (TLR-5). In another preferred variant the cell or cell line expresses at least the human TLR type 6 (TLR-6). In another preferred variant the cell or cell line expresses at least the human TLR type 7 (TLR-7). In another preferred variant the cell or cell line expresses at least the human TLR type 8 (TLR-8). In another preferred variant the cell or cell line expresses at least the human TLR type 9 (TLR-9). In another preferred variant the cell or cell line expresses at least the human TLR type 10 (TLR-10). In a preferred variant the cell or cell line expresses the at least heterodimeric receptor from human TLR type 1 (TLR-1) and human TLR type 2 (TLR-2). In another preferred variant the cell or cell line expresses the at least heterodimeric receptor from human TLR type 7 (TLR-7) and human TLR type 8 (TLR-8). In another preferred variant the cell or cell line expresses the at least heterodimeric receptor from human TLR type 6 (TLR-6) and human TLR type 2 (TLR-2). The invention also concerns co-expression of further TLRs chosen from the group consisting of TLR-1, TLR-2, TLR-3, TLR-4, TLR-5, TLR-6, TLR-7, TLR-8, TLR-9 and TLR-10. The invention preferably therefore concerns the following heterodimers: TLR-1/TLR-2; TLR1-TLR-3; TLR-1/TLR-4; TLR-1/TLR-5; TLR-1/TLR-6; TLR-1/TLR-7; TLR-1/TLR-8; TLR-1/TLR-9; TLR-1/TLR-10; TLR-2/TLR-3; TLR-2/TLR-4; TLR-2/TLR-5; TLR-2/TLR-6; TLR-2/TLR-7; TLR-2/TLR-8; TLR-2/TLR-9; TLR-2/TLR-10; TLR-3/TLR-4; TLR-3/TLR-5; TLR-3/TLR-6; TLR-3/TLR-7; TLR-3/TLR-8; TLR-3/TLR-9; TLR-3/TLR-10; TLR-4/TLR-5; TLR-4/TLR-6; TLR-4/TLR-7; TLR-4/TLR-8; TLR-4/TLR-9; TLR-4/TLR-10; TLR-5/TLR-6; TLR-5/TLR-7; TLR-5/TLR-8; TLR-5/TLR-9; TLR-5/TLR-10; TLR-6/TLR-7; TLR-6/TLR-8; TLR-6/TLR-9; TLR-6/TLR-10; TLR-7/TLR-8; TLR-7/TLR-9; TLR-7/TLR-10; TLR-8/TLR-9; TLR-8/TLR-10; TLR-9/TLR-10. These can be co-expressed alone or in combination at least with other TLR or TLR heterodimers.

However, the invention is not restricted to human TLRs. Especially for animal experimental applications and veterinary purposes, for treatment of infectious diseases in animals it is proposed that the cell according to the invention express animal TLR, preferably mammalian TLR. The TLR is chosen with particular preference from murine TLRs. In a preferred variant of the invention the cell or cell line expresses at least the murine TLR type 1 (mTRL-1). In another preferred variant the cell or cell line expresses at least the murine TLR type 2 (mTRL-2). In another preferred variant the cell or cell line expresses at least the murine TLR type 3 (mTRL-3). In another preferred variant the cell or cell line expresses at least the murine TLR type 4 (mTRL-4). In another preferred variant the cell or cell line expresses at least the murine TLR type 5 (mTRL-5). In another preferred variant the cell or cell line expresses at least the murine TLR type 6 (mTRL-6). In another preferred variant the cell or cell line expresses at least the murine TLR type 7 (mTRL-7). In another preferred variant the cell or cell line expresses at least the murine TLR type 8 (mTRL-8). In another preferred variant the cell or cell line expresses at least the murine TLR type 9 (mTRL-9). In another preferred variant the cell or cell line expresses at least the murine TLR type 10 (mTRL-10). In another preferred variant the cell or cell line expresses at least the murine TLR type 11 (mTRL-11). In another preferred variant the cell or cell line expresses at least the murine TLR type 12 (mTRL-12). In another preferred variant the cell or cell line expresses at least the murine TLR type 13 (mTRL-13).

In a preferred variant of the invention, not only a single transgenic cell or cell line according to the invention, which expresses specifically at least one TLR or TLR heterodimer is provided, which is referred to below as “cell type.” A “set” of at least two different cell types according to the invention, each of which expresses different TLRs or TLR heterodimers, is preferred. Sets of three, four, five, six, seven, eight, nine, ten or more different cell types according to the invention, each of which express different TLRs or TLR heterodimers, are particularly preferred. Without being bound to the theory, individual pyrogens of a pyrogen population each specifically bond to one or a few specific TLRs and/or TLR heterodimers. In this way a simple characterization of individual pyrogens and/or the pyrogen spectrum of the sample is possible. For example, a cell type that expresses the heterodimer from TLR-2 and TLR-6 permits specific detection of mycoplasma pyrogens and yeast pyrogens. For example, a set from a cell type that expresses TLR-3 and a cell type that expresses TLR-9 permits specific detection of viruses with double-stranded RNA.

An object of the invention is therefore also a cell culture vessel, preferably a cell culture plate, multiwell plate, in which at least one cell type, preferably several different cell types are introduced, adhered or incubated as a suspension. In the simplest case the cells lie on the surface of the cell culture vessel, for example, adhered to collagen film. However, culturing in suspension is preferred. Incubation/culturing on or in 3D biomatrices is also possible. It is also possible to introduce different cell types on or into addressable subcompartments of a cell culture support. The invention proposes to inoculate the cells on plates, vessels or wells and store them for further use, preferably freeze them or cryoconserve them. Plates, vessels or wells so prepared can then be used as required within a short time for corresponding tests for pyrogens and other TLR-activating or modulating substances (TLR antagonists). In the simplest case the plates, etc. with the cells are thawed, incubated with the sample being tested. The reporter gene-mediated enzyme activity is detected in known fashion and demonstrates in the cell type the presence or absence of a specific activation of TLR by the substance, pyrogen or PAMP being tested.

The cell culture vessel is preferably furnished in a kit. The kit contains the cells characterized above in a cell culture vessel already described or assay support and is preferably furnished in the frozen state especially for immediate performance of the test. During use of the kit costly cell culture conditions, like a CO₂ incubator, are advantageously unnecessary. The kit can be conducted in a simple laboratory of a hospital with devices for detection of the color change. In the simplest case the instantaneously recognizable color change is already sufficient for specific TLR activation. From the pattern of the color change the user of the kit can draw conclusions concerning the pyrogen spectrum and/or pathogen type.

The kit according to the invention for specific detection of a pyrogen in the sample contains at least one transgenic cell according to one of the preceding claims in a culture vessel and preferably detection medium, containing at least a substrate for the enzyme coded by the inducible reporter gene. A kit containing a cell culture vessel or plate with at least two compartments or wells is preferred, in which at least one transgenic cell, expressing at least a first TLR type or heterodimer is contained in a first well and a second transgenic cell different from the first transgenic cell, expressing at least a second TLR type or heterodimer is contained in a second well.

Accordingly, a particularly preferred variant of the invention proposes: a kit consisting of one or more cell culture vessels with a first well containing a first transgenic cell, preferably expressing at least the human TLR-1, a second well containing a second transgenic cell, preferably expressing at least the human TLR-2 and preferably a third well containing a third transgenic cell, preferably expressing at least the human TLR-3, preferably a fourth well containing a fourth transgenic cell, preferably expressing at least the human TLR-4, preferably a fifth well containing a fifth transgenic cell, preferably expressing at least the human TLR-5, preferably a sixth well containing a sixth transgenic cell, preferably expressing at least the human TLR-6, preferably a seventh well containing a seventh transgenic cell, preferably expressing at least the human TLR-7, preferably an eighth well containing an eighth transgenic cell, preferably expressing at least the human TLR-8, preferably a ninth well containing a ninth transgenic cell, preferably expressing at least the human TLR-9, preferably a tenth well containing a tenth transgenic cell, preferably expressing at least the human TLR-10, as well as preferably a detection medium.

Another object of the invention is also a method for specific detection of a pyrogen in the sample. According to the invention the method includes at least the steps: preparation of a sample, preparation of at least one transgenic cell or cell line according to the invention, which expresses at least one specific TLR or a specific TLR heterodimer; bringing the sample into contact with the cell so that a so-called sample-cell complex is formed, which is characterized in particular by bonding of sample components to the cell; incubation of the sample-cell complex for induction of enzyme activity, preferably at about 37° C. for about 3 to about 24 hours and detection of the enzyme activity induced by the reporter gene, in which the enzyme activity indicates the presence of a pyrogen specific for the TLR type or TLR heterodimer of the cell or cell line or agonistic active ingredient.

Detection of the enzyme activity induced by the reporter gene preferably occurs by furnishing a detection medium, containing a substrate for the enzyme coded by the inducible reporter gene of the cell and by incubation of the induced sample-cell complex in the detection medium, preferably at about 37° C. and preferably for about 30 to about 240 minutes, in which the enzyme activity is detected and preferably quantified by detection of the enzymatically converted substrate. Quantification of the enzymatically converted substrate and therefore the enzyme activity permits conclusions concerning the activity and concentration of the specific pyrogen or TLR-activating substance (TLR agonist; CpG motif, etc.).

The enzyme activity is preferably an alkaline phosphatase activity which is preferably mediated by SEAP. Alkaline phosphatases are enzymes that catalyze hydrolysis of phosphoric acid esters in an alkaline medium. 5-Bromo-4-chloroindolyl phosphate (BCIP) is preferably used as substrate. Detection of enzyme activity then occurs by the blue color change and/or blue precipitate, a dark blue-colored, insoluble and readily recognizable precipitate of indigo.

In an alternative variant the substrate is p-nitrophenyl phosphate (pNPP) and the alkaline phosphatase activity is indicated by hydrolytic cleavage of pNPP by the yellow color change of the solution. The yellow color change of the solution is preferably detected and quantified photometrically. Photometric analysis preferably occurs at about 405 nm. The concentration of pyrogen or TLR-activating substance in the sample can be determined from the extinction. In order to be able to also quantify reporter genes that are detected intracellularly, the cells must be lysed and the dye released from them. Direct intracellular detection occurs by dissolution of the dye in the cells via NaOH; intracellular quantitative measurements are possible on this account. A densitometric evaluation (via half-tones) is naturally also possible for quantification.

In another preferred variant the enzyme activity is a β-galactosidase activity and the substrate is preferably 5-bromo-4-chloro-3-indolyl-β-D-galactopyranoside (X-Gal). Detection of enzyme activity occurs by the blue color change and/or blue precipitate.

In another preferred variant the enzyme activity is luciferase activity and the substrate is preferably luciferin. In the presence of optionally additionally added ATP and Mg²⁺ the enzyme activity is indicated by luminescence (chemiluminescence assay).

The sample, which can be analyzed by the method or test system according to the invention, is especially a clinical sample from a human or animal body. The sample is preferably blood, preferably whole blood, for example, in the case of sepsis. Other clinical samples are blood serum, blood plasma, urine, sputum, stool, tissue biopsy, bronchial lavage, CNS fluid, CSF, lymph, synovial fluid and the like, for example, for typing of infection. Another object of the invention is therefore use of the transgenic cell for specific detection of a pyrogen in a clinical sample, preferably according to the method of the invention and/or preferably using the kit according to the invention.

It has surprisingly been shown that the transgenic cell or cell line according to the invention can be used in a test system in order to test products for apyrogenicity. If the method for testing for apyrogenicity or determination of pyrogen contamination is used, the sample is preferably a test piece (specimen) of a medical instrument or medical product (MP) or in vitro diagnostic agent (IVD) or a drug, drug ingredient, food, food ingredient or raw material or starting material for foods or drugs. These include surgical instruments, cannulas, syringes, infusion sets, blood bags and transfusion sets, dialysis sets and equipment, wound coverings, suture material, implants, prostheses, catheters, infusion solutions, rinsing solutions and the like. It is also proposed that the sample be chosen from transplants, tissues and cells of human origin and products of this content or this origin, as well as transplants, tissues, cells of animal origin and products of this content or this origin. It is also proposed that the sample be chosen from cosmetic articles and cosmetics. Another object of the invention is therefore the use of the transgenic cell for testing of such products for apyrogenicity, preferably according to the method of the invention and/or preferably using the kit according to the invention.

It has also surprisingly been found that the transgenic cell or cell line according to the invention can be used in a test system in order to find active ingredients with the property of a TLR antagonist in a group of candidate substances and to quantify their efficacy. Another object of the invention is therefore use of the transgenic cell for screening of active ingredients with the property of a TLR antagonist, preferably according to the method of the invention and/or preferably using the kit according to the invention.

Finally, it has surprisingly been found that the transgenic cell or cell line according to the invention could be used in a test system in order to find oligonucleotides with CpG motifs that activate specific TLR, especially TLR-9, of a group of candidate substances and to quantify their efficacy. Another object of the invention is therefore finally use of the transgenic cell for screening of oligonucleotides with CpG motifs, preferably according to the method of the invention and/or preferably using the kit according to the invention.

### BRIEF DESCRIPTION OF THE DRAWINGS

FIG. 1 shows a schematic view of the test method according to the invention (on the example of TLR-4/CD14 (MD2) with the ligands LPS).

FIG. 2 shows NIH-3T3 clone 4/5 TLR-4/CD14 with SEAP reporter plasmid after thawing and after addition of 100 ng/mL LPS (4-well right side) and detection medium with SCIP substrate.

FIG. 3 shows induction of NIH-3T3 TLR-4/CD14 test system with 10 pg/mL to 100 pg/mL LPS; substrate: BCIP; negative control was not induced or induced with ssRNA40.

FIG. 4 shows sensitivity detection of NIH-3T3 clone 4/5 TLR-4/CD14; LPS specifically to 10 pg/mL, 2 hours after addition of detection medium: photometric analysis.

FIG. 5 shows sensitivity detection of NIH-3T3 clone 4(5) TLR-4/CD14; LPS is specifically detectable to 1 pg/mL, 2 hours after addition of detection medium: photometric analysis.

FIGS. 6 and 7 comparative experiment: TLR-4 test with HEK blue 293 fibroblasts and other 293 fibroblasts, transfected with TLR-4/CD14 SEAP, induced with 100 ng/mL LPS; both the induced and the noninduced control show a blue color change; a specific detection is not possible with these cells.

FIG. 8 comparative experiment: TLR-4 test with HEK blue 293 fibroblasts and other 293 fibroblasts transfected with TLR-4/CD14 SEAP induced with 100 ng/mL LPS: SDS-PAGE/Western Blot analysis of cell pellets, primary antibodies: anti-SEAP as well as anti-mouse POD conjugated secondary antibodies, markers: SeeBlue® Plus2 prestained, standard: alkaline phosphatase (SEAP); in HEK 293 and other 293 cells (K2 and K4) both in the induced cells and in the noninduced control cells expressed in the same amount; specific detection is not possible with these cells.

FIG. 9 shows specificity of the test system: NIH-3T3 TLR-4/CD14 test system was induced with nonspecific pyrogens (each 25 μg/mL); ODN (ligand for TLR-9), PGN (ligand for TLR-2), Poly IC; no color change occurs, the test reacts specifically.

FIG. 10 shows phase contrast recording of NIH-3T3 TLR-4/CD 14 clone 4/5: 30,000 cells/well inoculated, 100 μL in 30% FCS, 80 mmol/L HEPES and 5% DMSO, frozen for 3 days to 4 weeks at 80° C.; adhesion overnight 37° C., humid atmosphere without CO₂.

FIG. 11 shows TLR-4 test according to the invention conducted on frozen and rethawed NIH-3T3 TLR-4/CD14 SEAP P40 induced with 100 pg/mL LPS and 100 pg/mL ssRNA40, induction after 24 hours, detection after 3 hours; specific blue coloration of the induced cells is observed.

FIG. 12 shows TLR-5 test according to the invention conducted on NIH-3T3 clones TLR-5 SEAP induced with 2 μg/mL flagellin; specific blue coloration or the induced cells is observed.

FIG. 13 comparative experiment: TLR-5 test with HELA cells transfected with TLR-5 SEAP induced with 2 μg/mL flagellin; both the induced cells and the noninduced controls show intense blue coloration. No specific induction is possible with this cell line.

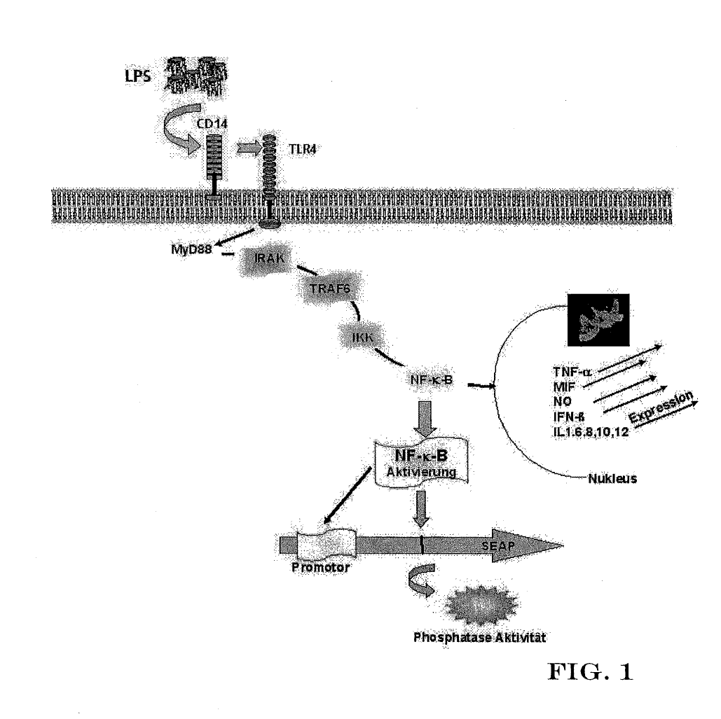

### PRACTICAL EXAMPLES

### Example 1

### TLR-4 Specific Test System

### Methods

Transfection with TLR-4

The cell line NIH-3T3 was transfected with a TLR-4/CD14 complex as well as the reporter gene plasmid SEAP/ELAM-1.

The endotoxin (LPS)-mediated induction of TLR-4 leads according to a signal cascade to activation of the transcription factor NF-κB. Expression of the reporter gene SEAP is controlled by an ELAM-1 promoter inducible by NF-κB. NF-κB activation and therefore specifically secretion of SEAP then occurs on induction of TLR-4 by the endotoxin lipopolysaccharide LPS.

### Performance of the Test

NIH-3T3 TLR-4/CD14 clone 4/5 P35 in a density of 30,000 to 200,000 cells/well (24-well) (corresponding cell count for other well volume) are inoculated in 500 μL/well in 0.5% FCS medium o/n for adhesion.

On the next day induction occurs with LPS o/n (+negative control: ssRNA40 cannot be detected by TLR-4/CD14).

On the next day detection occurs by incubation with 300 μL/well detection medium, which is added directly to the induced cells: in the induced cells SEAP activity converts the substrate BCIP in the detection medium to a dark blue insoluble end product (indigo). As an alternative SEAP activity in the induced cells converts the substrate pNPP in the detection medium to a light yellow soluble color complex, which is determined photometrically at about 405 nm. The photometric analysis occurs about 2 hours after addition of the detection medium.

Freezing and Use of Test Kits with 24-Well Plate

In one variant transfected cell lines are frozen in the corresponding assay format, here: a cell culture-well-plate (multiwell plate) in corresponding density (200,000 cells/well in 500 μL each (in 24-well)).

The user receives the assay kit with 8 to 10 different cell lines in corresponding medium already in the test plate delivered cooled on dry ice. The assay can be taken from the package and incubated directly in a 37° C. cabinet.

After thawing of the kit, addition of DMEM culture medium occurs for adhesive of the cells overnight. A medium with HEPES buffer permits incubation of the cell test in a heating cabinet, i.e., without CO₂ gassing. Induction is carried out per test by addition of 100 ng/mL LPS. The detection is conducted after 24 hours by addition of 300 μL/well BCIP detection medium.

### Results

### a) Thawed Test System

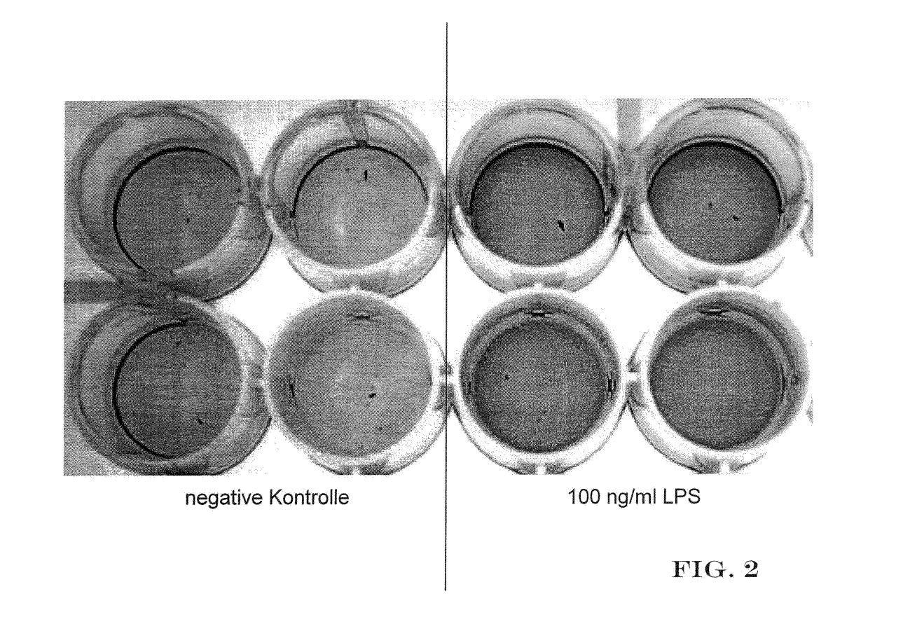

FIG. 2 shows the results of negative control and with 100 ng/mL LPS on 3T3 NIH clone 4/5 TLR-4/CD14 after thawing. In the induced cells SEAP activity converts the substrate BCIP in the detection medium to a deep dark blue insoluble end product (indigo).

A rapid, simple detection system that can be operated without large equipment expense and cell culture laboratory (sterile bench and CO₂ incubator, etc.) was developed for LPS based on cells that were stably transfected with TLR-4/Cd14. It could be performed rapidly and is simple to handle.

### b) Sensitivity

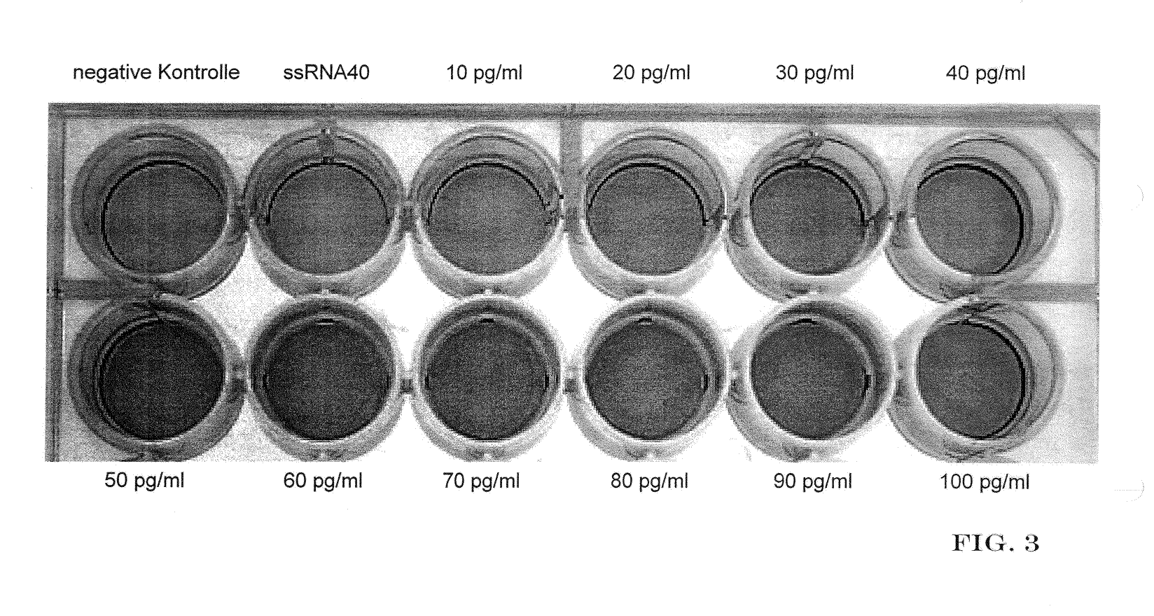

FIG. 3 shows induction of the NIH-3T3 clone 4(5) TLR-4/CD14 SEAP test system with 10 pg/mL to 100 pg/mL LPS. The substrate of the detection medium is BCIP. A negative control was not induced, another negative control was induced with ssRNA40 nonspecifically.

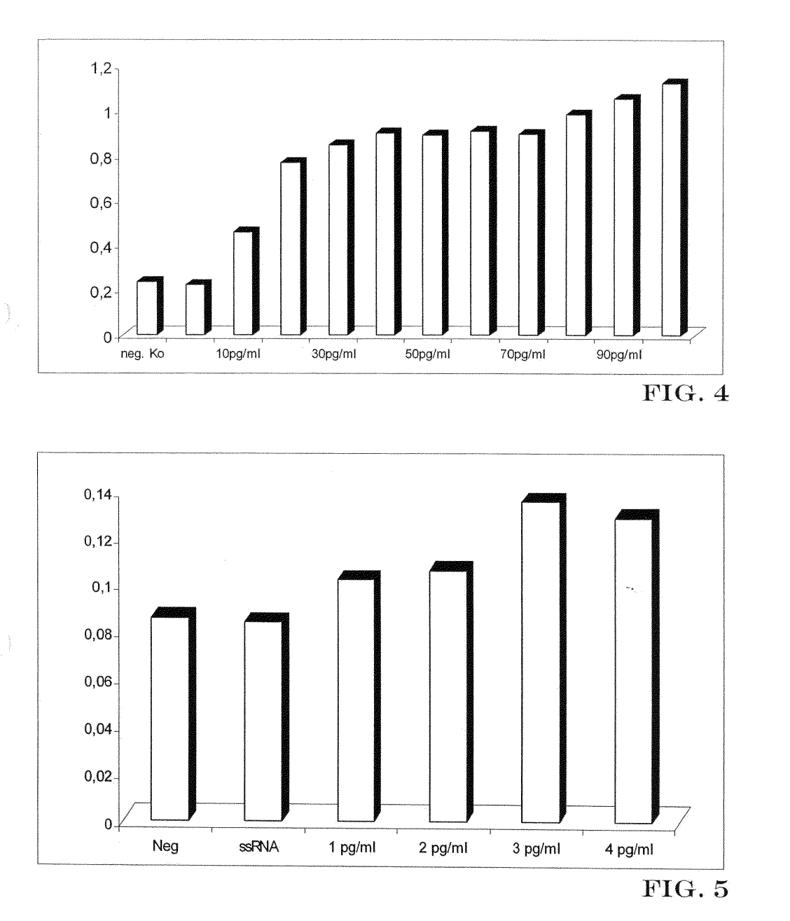

FIG. 4 shows the sensitivity detection of NIH-3T3 clone 4(5) TLR-4/CD14 SEAP specifically to 10 pg/mL LPS. FIG. 5 shows the sensitivity detection of NIH-3T3 clone 4(5) TLR-4/CD14 SEAP specifically to 1 pg/mL LPS.

The sensitivity of the test system lies at about 1 to 2 pg/mL LPS.

### c) Specificity

The aforementioned HIH-3T3 TLR-4/CD14 SEAP test system was induced with large amounts of nonspecific pyrogen (25 μg/mL ODN, PGN, Poly IC each) for which TLR-4 does not bond: ODN, PGN, Poly IC were recognized by TLR-9, 2 and 3 but not by TLR-4. FIG. 5 shows the result: even with extremely high nonspecific pyrogen fraction no color change can be seen in the TLR-4-specific system; the TLR-4 test is specific.

### Example 2

### Comparative Experiments

### Method

HEK blue 293 fibroblasts and other 293 fibroblasts were transfected with TLR-4/CD14 SEAP and induced with 100 ng/mL LPS. All other process parameters were chosen as in example 1 according to the invention.

In addition, a Western Blot analysis of gene expression was conducted in known fashion: first antibody: anti-SEAP; conjugated second antibody: anti-mouse POD; marker: SeeBlue® Plus2; prestained standard.

### Results

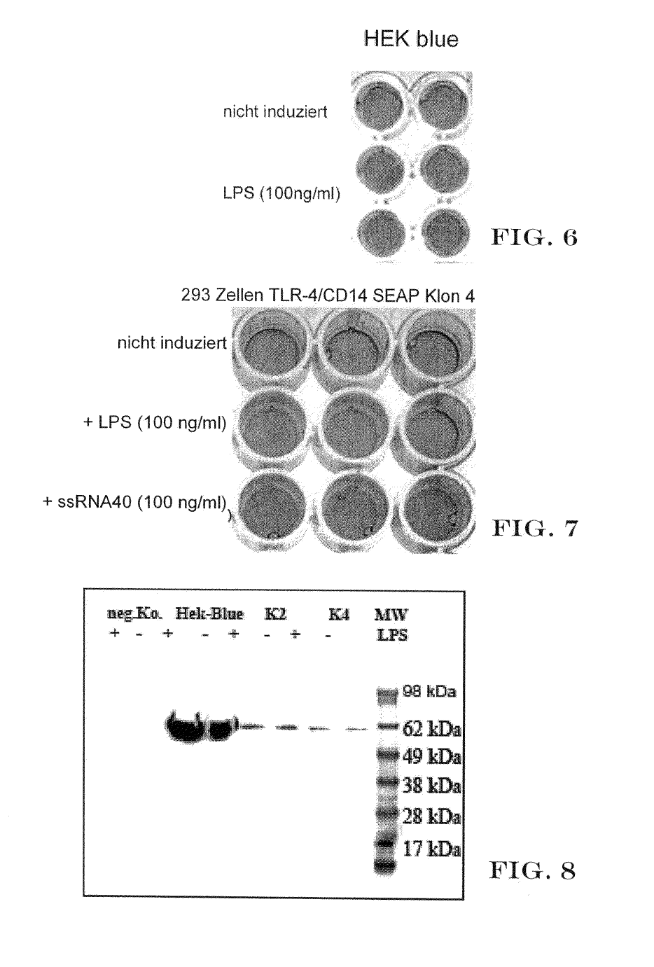

FIGS. 6 and 7 show the results of the color test: not only the induced cells but also the noninduced controls show a blue color change. With HEK blue 293 and other 293 cells like clone 4(K4) no specific induction and therefore no establishment of the test system is possible.

FIG. 8 shows the results of SDS-PAGE/Western Blot analysis of the cell pellets after induction with 100 ng/mL LPS: the alkaline phosphatase is expressed in the HEK293 and in other 293 cells (K2 and K4), in the noninduced control cells in the same amount as in the induced cells; a specific detection of TLR-activating substances, pyrogens, PAMPs is not possible with these cells.

### Example 3

### Assay in 96-Well Scale and CO₂-Free Culturing

### Method

On a multiwell plate (96-well) NIH-3T3 TLR-4/CD14 SEAP P40 cells were frozen in a density of 30,000 cells/well in 100 μL medium (DMEM 80 mmol/L HEPES, 30% FCS and 5% DMSO) in suspension at −80° C.

After 72 hours to 4 weeks at −80° C. the cells were thawed by addition of 100 μL medium (10% FCS) at 37° C. CO₂-free.

After 24 hours adhesion was changed to medium (DMEM 0.5% FCS) and the cells induced in 100 μL with 30 pg/mL LPS or 30 pg/mL ssRNA33 (control).

After 24 hours, a media change to detection medium was conducted. As an alternative 100 μL/well detection medium is added to the well directly after the induction time.

### Results

FIG. 11 shows the results: after 3 to 24 hours at the latest the detection of the induced enzyme activity is distinct. If detection medium is added directly to the well after the induction time, a signal is detectable after 1 to 3 hours.

### Example 4

### Histology in CO₂-Free Culturing

### Method

On a multiwell plate NIH-3T3 TLR-4/CD14 SEAP cells were inoculated in a density of 30,000 cells/well in 100 μL medium (30% FCS, 80 mmol/L HEPES). Adhesion occurred overnight at 37° C. in a humid atmosphere without CO₂.

### Results

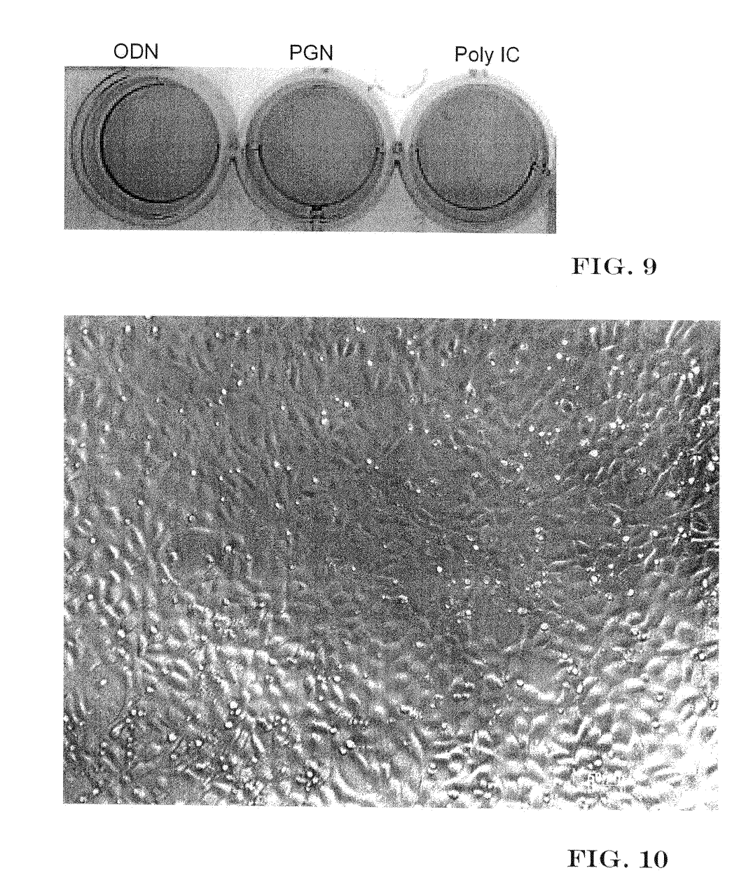

FIG. 10 shows the phase contrast recording of the adhered cells in the well: the cells grow during culturing in a 37° C. heating cabinet in a HEPES-buffered medium. The figure shows an intact cell monolayer.

### Example 5

### TLR-5-Specific Test System

### Method

Transfection with TLR-5

The cell line NIH-3T3 was transfected with TLR-5 and the reporter gene plasmid SEAP. The measures correspond to example 1.

### Performance of the Test

NIH-3T3 clone TLR-5 was inoculated in a density of 30,000 to 200,000 cells/well (24-well) in 500 μL/well in 0.5% FCS medium; induction occurs with 2 μg/mL flagellin; detection by incubation with 300 μL/well detection medium with BCIP.

### Results

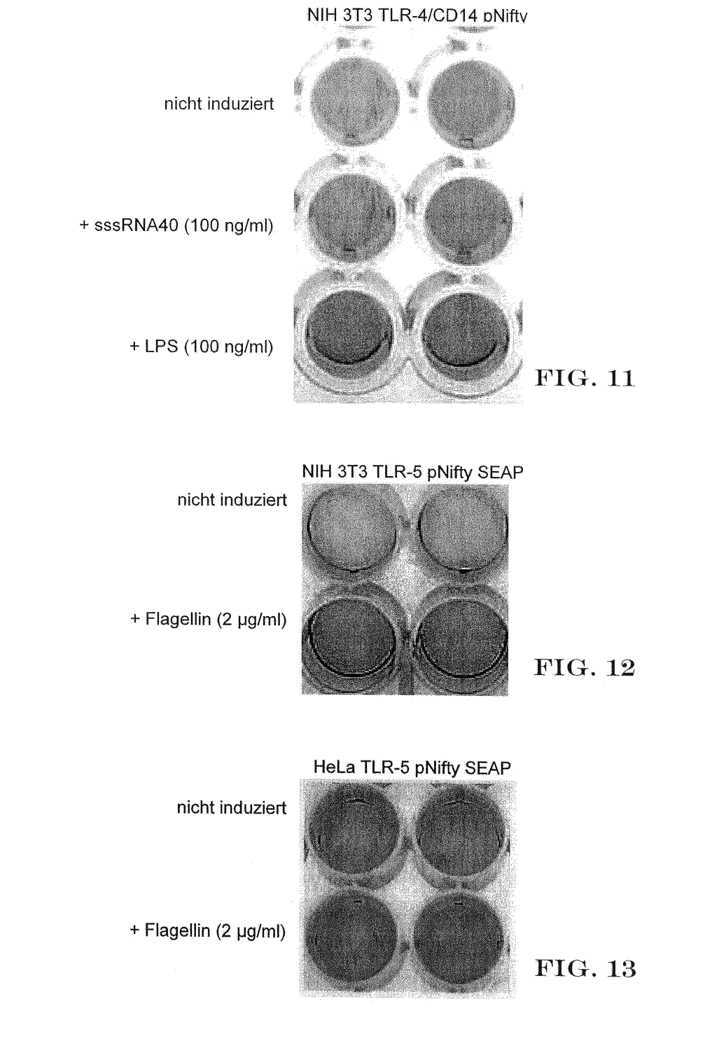

FIG. 12 shows the results of the color test: a specific blue coloration of the induced cells occurs; noninduced control cells show no blue coloration.

1. Transgenic cell for specific detection of a pyrogen in a sample containing in the genome:
a) a gene or genes that code for at least one toll-like receptor (TLR) and
b) at least one reporter gene under the expression control of a promoter inducible by
NF-κB.

2. Cell according to claim 1, in which the cell is a mammalian fibroblast cell.

3. Cell according to claim 2, in which the cell is a murine fibroblast cell of type NIH-3T3.

4. Cell according to one of the preceding claims, in which the cell contains a gene or genes that code for a first toll-like receptor type (TLR type) and additionally a gene or genes that codes for second toll-like receptor type (TLR type).

5. Cell according to one of the preceding claims, in which the cells additionally have a gene that codes for the CD14 receptor.

6. Cell according to claim 5, in which the CD14 receptor is co-expressed in combination with human TLR type 4 (TLR-4).

7. Cell according to one of the preceding claims, in which the reporter gene codes for a secreted alkaline phosphatase (SEAP).

8. Cell according to one of the preceding claims, in which the reporter gene codes for a β-galactosidase.

9. Cell according to one of the preceding claims, in which the reporter gene codes for a luciferase.

10. Cell according to one of the preceding claims, in which the reporter gene codes for GFP.

11. Cell according to one of the preceding claims, in which the inducible promoter is the promoter for selectin (endothelial cell leukocyte adhesion molecule; ELAM-1).

12. Cell according to one of the preceding claims, which expresses the human TLR type 1 (TLR-1).

13. Cell according to one of the preceding claims, which expresses the human TLR type 2 (TLR-2).

14. Cell according to one of the preceding claims, which expresses the human TLR type 3 (TLR-3).

15. Cell according to one of the preceding claims, which expresses the human TLR type 4 (TLR-4).

16. Cell according to one of the preceding claims, which expresses the human TLR type 5 (TLR-5).

17. Cell according to one of the preceding claims, which expresses the human TLR type 6 (TLR-6).

18. Cell according to one of the preceding claims, which expresses the human TLR type 7 (TLR-7).

19. Cell according to one of the preceding claims, which expresses the human TLR type 8 (TLR-8).

20. Cell according to one of the preceding claims, which expresses the human TLR type 9 (TLR-9).

21. Cell according to one of the preceding claims, which expresses the human TLR type 10 (TLR-10).

22. Cell according to one of the preceding claims, which expresses the heterodimeric receptor for the human TLR type 1 (TLR-1) and human TLR type 2 (TLR-2).

23. Cell according to one of the preceding claims, which expresses the heterodimeric receptor for the human TLR type 6 (TLR-6) and human TLR type 2 (TLR-2).

24. Cell according to one of the preceding claims, which expresses the heterodimeric receptor for the human TLR type 7 (TLR-7) and human TLR type 8 (TLR-8).

25. Cell according to one of the preceding claims, which additionally expresses the coreceptor type CD14 (MD2).

26. Kit for specific detection of a pyrogen in the sample containing:
a culture vessel with at least one transgenic cell according to one of the preceding claims.

27. Kit according to claim 26 containing:
detection medium containing a substrate for the enzyme coded by the inducible reporter gene.

28. Kit according to claim 26 or 27 containing:
cell culture vessel or plate with at least two compartments or wells, in which at least a first transgenic cell that expresses at least the first TLR type is contained in a first well and a second transgenic cell different from the first transgenic cell that expresses at least a second TLR type is contained in a second well.

29. Kit according to claim 28 containing at least one well containing a transgenic cell that expresses the human TLR-1.

30. Kit according to claim 28 or 29 containing at least one well containing a transgenic cell that expresses the human TLR-2.

31. Kit according to one of the claims 28 to 30 containing at least one well containing a transgenic cell that expresses the human TLR-3.

32. Kit according to one of the claims 28 to 31 containing at least one well containing a transgenic cell that expresses the human TLR-4.

33. Kit according to one of the claims 28 to 32 containing at least one well containing a transgenic cell that expresses the human TLR-5.

34. Kit according to one of the claims 28 to 33 containing at least one well containing a transgenic cell that expresses the human TLR-6.

35. Kit according to one of the claims 28 to 34 containing at least one well containing a transgenic cell that expresses the human TLR-7.

36. Kit according to one of the claims 28 to 35 containing at least one well containing a transgenic cell that expresses the human TLR-8.

37. Kit according to one of the claims 28 to 36 containing at least one well containing a transgenic cell that expresses the human TLR-9.

38. Kit according to one of the claims 28 to 37 containing at least one well containing a transgenic cell that expresses the human TLR-10.

39. Kit according to one of the claims 28 to 38 containing at least one well containing a transgenic cell that expresses the heterodimer of human TLR-2 and human TLR-6.

40. Kit according to one of the claims 28 to 39 containing at least one well containing a transgenic cell that expresses the heterodimer of human TLR-2 and human TLR-1.

41. Kit according to one of the claims 28 to 39 containing at least one well containing a transgenic cell that expresses the heterodimer of human TLR-7 and human TLR-8.

42. Method for specific detection of a pyrogen in a sample comprising the steps:
a) Preparation of a sample of at least one transgenic cell according to one of the claims 1 to 25 that expresses at least one specific toll-like receptor (TLR) or a specific TLR heterodimer,
b) Bringing the sample in contact with the cell,
c) Incubation of the sample-cell complex to induction, and
d) Detection of the enzyme activity mediated by the induced reporter gene, in which detection of the enzyme activity indicates the presence of a pyrogen specific for the TLR type or TLR heterodimer.

43. Method according to claim 42, in which step d) includes the steps:
d1) Preparation of the detection medium containing substrate for the enzyme coded by the inducible reporter gene of the cell and
d2) Incubation of the induced sample-cell complex in the detection medium for detection of the enzyme activity mediated by the induced reporter gene.

44. Method according to claim 42 or 43, in which the enzyme activity is alkaline phosphatase activity.

45. Method according to claim 44, in which the substrate is 5-bromo-4-chloroindolyl phosphate (BCIP) and detection is indicated by blue color change and/or blue precipitate.

46. Method according to claim 45, in which the substrate is p-nitrophenyl phosphate (pNPP) and detection is indicated by yellow color change of solution.

47. Method according to claim 46, in which the yellow color change of the solution is quantified photometrically.

48. Method according to claim 42 or 43, in which the enzyme activity is β-galactosidase activity.

49. Method according to claim 48, in which the substrate is 5-bromo-4-chloro-3-indolyl-β-D-galactopyranoside (X-Gal) and detection is indicated by blue color change and/or blue precipitate.

50. Method according to claim 42 or 43, in which the enzyme activity is luciferase activity.

51. Method according to claim 50, in which the substrate is luceriferin, optionally with ATP and Mg²⁺ and detection is indicated by luminescence.

52. Method according to claim 42 or 43, in which the enzyme activity is GFP.

53. Method according to one of the claims 42 to 50, in which the sample is a clinical sample from a human or animal body.

54. Method according to claim 53, in which the sample is blood.

55. Method according to one of the claims 42 to 50, in which the sample is a specimen of a medical instrument or medical product.

56. Method according to one of the claims 42 to 50, in which the sample is a drug, drug ingredient, food, food ingredient or raw material or starting material for foods or drugs.

57. Method according to one of the claims 42 to 56, in which in step c) incubation of the sample-cell complex for induction occurs at about 37° C. for at least about an hour and a maximum of about 24 hours.

58. Use of the transgenic cell according to one of the claims 1 to 25 for specific detection of the pyrogen in a clinical sample.

59. Use of the transgenic cell according to one of the claims 1 to 25 for testing of products for apyrogenicity.

60. Use of the transgenic cell according to one of the claims 1 to 25 for screening of active ingredients with the property of a TLR antagonist.

61. Use of the transgenic cell according to one of the claims 1 to 25 for screening of oligonucleotides with CpG motifs.

62. Use according to one of the claims 58 to 61, in which the kit is used according to claims 26 to 41.

63. Use according to one of the claims 58 to 61, in which the method is conducted according to one of the claims 42 to 57.
